(19) United States
(12) Patent Application Publication
(10) Pub. No.: US 2026/0297480 A1
(43) Pub. Date: Oct. 1, 2026

(54) METHOD FOR CONTROLLING BIOGAS PRODUCTION PROCESSES AND DIGESTER FOR BIOGAS PRODUCTION

(71) Applicant: MAPRO S.p.A, NOVA MILANESE (MONZA BRIANZA) (IT)

(72) Inventors: CARMINE MADDALENA, PADERNO DUGNANO (IT); PAOLO CEOLA, MILANO (IT); VALTER MASSARI, LISSONE (IT)

(21) Appl. No.: 19/490,071

(22) PCT Filed: Apr. 23, 2024
(86) PCT No.: PCT/IB2024/053936

§ 371 Date: Dec. 4, 2025

(30) Foreign Application Priority Data
Jun. 6, 2023 (IT) 102023000011457

(51) Int. Cl. C12M 1/107; C12M 1/00; C12M 1/36

(52) U.S. Cl. CPC C12M 21/04; C12M 29/24; C12M 41/48

(57) ABSTRACT

A method for the optimisation of the gas injection system and for the control of biogas production processes including a step of detecting a working parameter of the biogas production process and a step of driving the biogas production process based on real-time processing of the working parameter.

The present invention relates to a method for the optimisation and control of biogas production processes.

The present invention also provides a digester for the purification and production of biogas from sludge that is particularly suitable for operating this method.

In other words, the present invention fits into the field of purification plants and biogas production from sludge, e.g. of municipal origin, from agricultural products or waste, or from animal husbandry and the food industry.

Generally, it is known to purify and produce biogas from sludge using digesters suitable for containing a volume of sludge and producing a biogas from that volume of sludge.

Mixing of the sludge contained within the digester is known in order to optimise biogas production while preventing damage to the plant and the digester itself.

Mechanical systems are now used to perform such mixing, which are immersed in the sludge with the consequent problem of damage, such as shovels or recirculation pumps, or gas injection systems with external machines, such as lance mixing systems or venturi systems.

Control methods for such mixing systems are known, but they involve binary control, i.e. control aimed solely at activating and deactivating the mechanical or gas-driven systems in use for mixing the sludge.

Specifically, these methods involve monitoring one or more of the digester's operating parameters and if they assume values outside predetermined operating ranges, the digester, or even one or more of its components, is deactivated.

In known mixing systems, regulation of the mixing process is left to experienced operators.

In other words, known control methods suffer from the drawback of not providing for continuous adjustment of the working parameters of the systems in use to mix the sludge, resulting in little or no uniform mixing of sludge.

This results in both lower biogas production and higher digester maintenance costs.

Similarly, even during the digester production phase, the parametrisation of its structural characteristics is carried out in a rather onerous (in terms of time and cost) and random manner by an operator who has to rely mainly on his experience and knowledge.

As a result, the efficiency of known digesters, both in terms of their design and operation, is strongly influenced by the level of experience of the individual operators, resulting in digesters that are not always efficient and whose operation is not optimised.

Operationally, a digester exploits bacteria capable of digesting sludge anaerobically, producing biogas; however, these bacteria require a constant environment at a predetermined temperature to perform their function optimally.

Poor sludge mixing results in a high temperature gradient within the sludge in the digester. In other words, the sludge contained in different portions of the digester has different respective temperatures, disadvantageously causing a significant decrease in biogas production by the bacteria.

Poor sludge mixing also leads to sludge settling at the bottom of the digester. Sedimentation of sludge limits a digester's filling volume over time, affecting its efficiency.

Settled sludge can also stratify and solidify, leading to the formation of surface crusts within the digester, which must be removed (during routine or extraordinary maintenance) both to avoid a limitation of the digester's filling volume and to prevent possible damage by mechanical failure of parts of the digester itself.

In addition, settled sludge can lead to the formation of struvite, a composite material that tends to damage moving equipment installed in contact with the sludge, on which it settles, tending to block and damage it.

Inefficient sludge mixing not only causes a decrease in biogas production by the digester, but also causes an increase in the maintenance costs of the digester itself.

In addition, the energy consumption of known mixing systems is considerable, thus affecting the economic/environmental sustainability of the known digesters themselves.

In this context, the technical task underpinning the present invention is to propose a digester for the purification and production of biogas from sludge and a method for controlling biogas production processes that overcome the drawbacks of the above-mentioned prior art.

An object of the present invention is to propose a method for controlling biogas production processes that can maximise and optimise biogas production.

A further object of the present invention is to propose a method for controlling biogas production processes from sludge and a method for controlling biogas production processes that can prevent limitation of a digester filling volume during its service life.

A further object of the present invention is to propose a method for controlling biogas production processes that ensures contained maintenance costs.

Further, an object of the present invention is to propose a method for controlling biogas production processes that can ensure a more economically and environmentally sustainable sludge digestion process.

In addition, a further object of the present invention is to propose a digester capable of operating the method for controlling biogas production processes covered by the present invention.

The stated technical task and specified object are essentially achieved by a method for controlling biogas production processes.

The method comprises a step of providing a sequential biogas injection system inside a digester comprising a containment volume designed to contain sludge, and a step of filling the containment volume with sludge.

The method involves a step of sucking biogas produced and/or contained in the sludge from the containment volume, and a step of injecting at least a part of sucked biogas into the containment volume, in detail through injection portions of the recirculation element.

The method also comprises steps aimed to control the sucking and/or injection of biogas.

In detail, the method comprises a step of detecting at least one working parameter of the digester by means of at least one detector device and a step of transmitting the working parameter to a control unit.

The method also includes a step of processing the working parameter via the control unit and driving, via the control unit itself, the suction step and/or the biogas injection step.

Advantageously, the present invention thus provides a method for controlling biogas production processes that can maximise a biogas production while maintaining process sustainability and contained costs.

In general, an innovative control logic is therefore proposed to ensure high levels of efficiency for optimal biogas production in real time and continuously.

Further features and advantages of the present invention will become clearer from the indicative, and therefore non-limiting, description of preferred but not exclusive embodiments of a method for controlling biogas production processes and a digester for purification and biogas production from sludge.

Such description will be set forth herein below with reference to the accompanying drawings, provided for merely indicative and therefore non-limiting purposes, wherein:

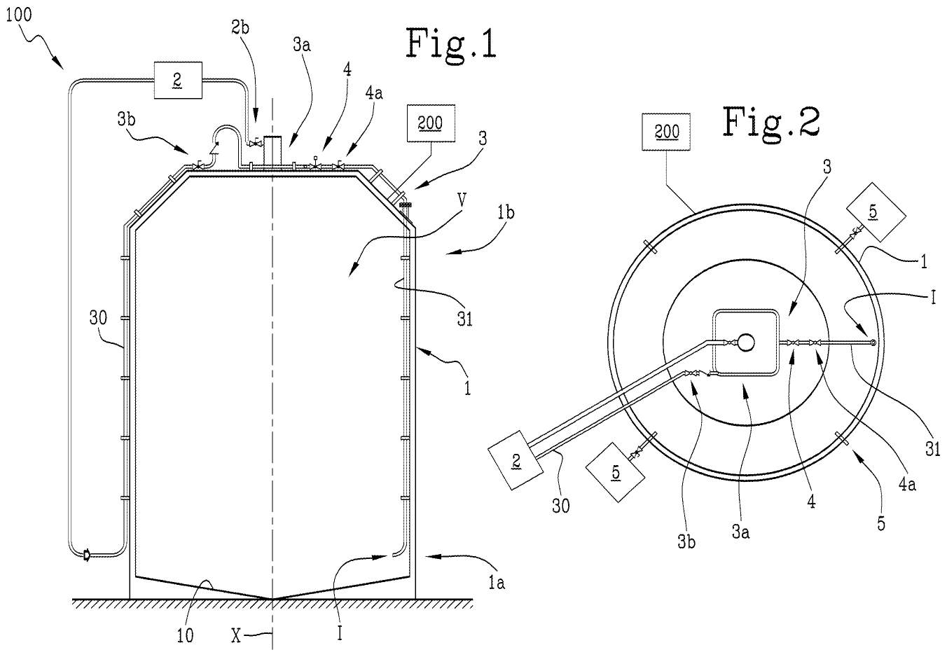

FIG. 1 shows a schematic front cross-sectional view of a digester according to the present invention;

FIG. 2 shows an upper schematic view of the digester in FIG. 1 with some components visible;

FIG. 3 shows a schematic side cross-sectional view of a digester according to the present invention;

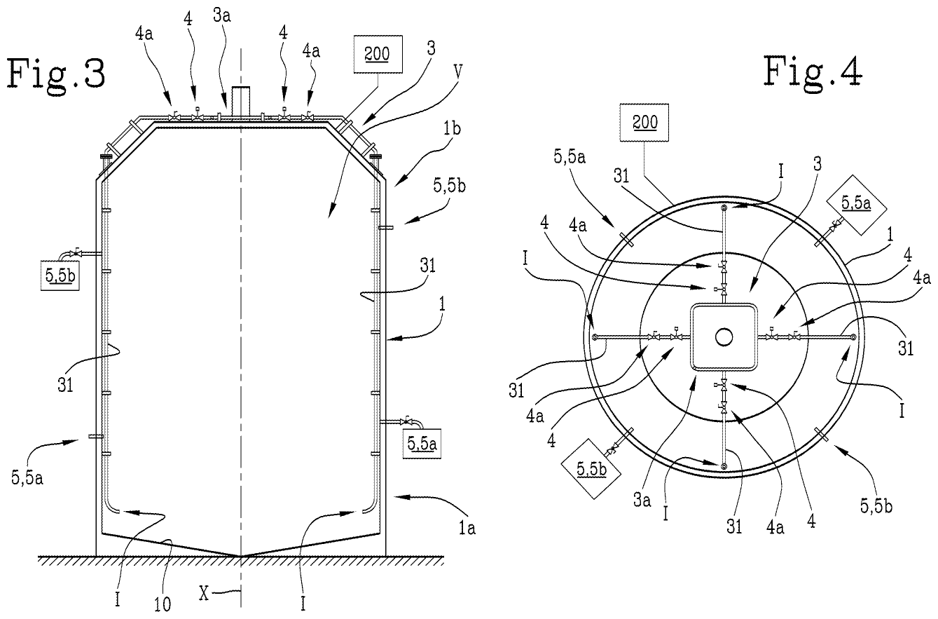

FIG. 4 shows an upper schematic view of the digester in FIG. 2 with some components visible;

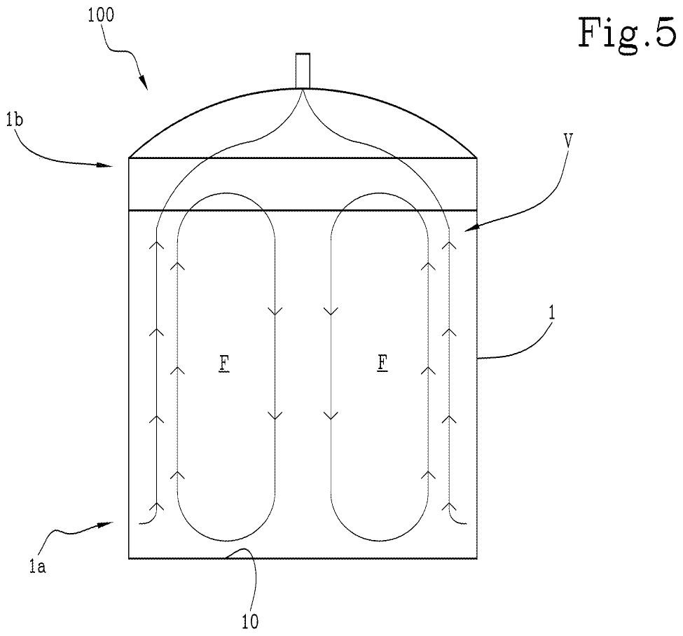

FIG. 5 is a schematic view of sludge mixing inside a digester according to the present invention.

With reference to the appended figures, the numerical reference 100 indicates a digester as a whole.

The digester 100 is suitable for the purification and production of biogas from sludge, e.g. of municipal, animal or agricultural origin.

In detail, the method according to the present invention is performed by setting up a digester, preferably the digester 100, internally defining a containment volume intended to contain sludge and biogas.

With reference to FIGS. 1 and 3, the digester 100 may comprise a containment body 1 internally defining a containment volume “V” intended to contain sludge and biogas.

The digester 100 can also have variable dimensions and a variable number of injection points.

The method also involves filling the containment volume “V” with sludge and sucking a fraction of the biogas produced and/or contained in the sludge from the containment volume “V”.

The method then involves injecting at least a part of the sucked biogas into the containment volume “V”, performing biogas recirculation within the digester.

The method also includes steps relating to control of the suction and/or injection steps, controlling the duration and/or positioning in relation to suction and injection.

In particular, at least one working parameter of the digester 100 is detected and transmitted to a control unit 200.

This working parameter can also be processed by means of the control unit 200, thus allowing the suction and/or injection of biogas to be driven, again by means of the control unit, on the basis of the processed working parameter.

Advantageously, the method thus makes it possible to monitor the operation of the digester 100 (by processing the working parameter detected by each detector device) and to drive the suction and/or injection of biogas on the basis of the processed working parameter, so as to strive for maximisation of biogas production, minimisation of energy consumption, or both.

In this context, the step of sucking the biogas from the containment volume “V” is preferably carried out by means of a compressor 2, while the step of injecting the biogas into the containment volume “V” is carried out by means of a recirculation element 3 including respective injection portions “I” suitable for injecting biogas into the containment volume “V”.

The step of detecting at least one working parameter is carried out by means of one or more detector devices 5, detecting one or more of: at least one compressor operating parameter 2, at least one chemical parameter of sludge and/or biogas contained within the containment volume “V”, at least one physical parameter of sludge and/or biogas contained within the containment volume “V”.

For example, the step of detecting the at least one working parameter of the digester 100 may be performed by detecting a temperature of the sludge contained therein, so as to drive the compressor 2 and/or the recirculation element 3 to maintain a temperature, and/or a temperature gradient, within the sludge below a threshold value. As a result, biogas production by the bacteria responsible for it can be maximised.

Alternatively or additionally, the step of detecting at least one working parameter of the digester can be performed by detecting an energy consumption parameter of the digester 100, i.e. of the compressor 2, in order to drive it to minimise its energy consumption. Accordingly, the present invention provides a method for controlling sustainable biogas production processes from an environment and cost-related point of view.

In general, the detected working parameter is transmitted to the control unit 200, which is capable of processing the working parameter and executing the step of driving the injection of biogas.

Preferably, the method comprises a step of driving the suction of biogas from the containment volume “V”.

Preferably, the method also comprises initial steps related to a design of the biogas production process operated by the method.

In detail, the method comprises a step of acquiring information on the sludge to be treated by the digester 100.

For example, this information may comprise one or more of: a type of sludge intended to be contained within the containment volume “V”, a volume of sludge intended to be contained and processed within the containment volume “V”.

The term “type of sludge” can be understood as qualitative information, e.g. on the chemical composition of the sludge, its origin.

Based on this information, the method involves setting a technical characteristic of the digester 100.

The technical characteristic of the digester 100 may be related to a number of injection portions “I”, or to a mutual positioning of the injection portions “I” or to a positioning of the injection portions “I” within the containment volume “V”.

In other words, the method involves a step of designing the digester 100, in particular with respect to its structural characteristics (volume or shape, number and position of its components), based on the information on the sludge that this digester 100 is to process.

In addition, the method preferably involves storing a sequence of commands and/or movements in the control unit 200, based on the sludge information acquired.

In particular, the sequence of commands is suitable to move at least one injection valve 4 of the recirculation element 3 according to a predefined sequence of movements between an opening configuration and a closing configuration.

In more detail, the injection valve in the opening configuration allows an injection of biogas through the respective injection portion “I”, while in the closing configuration it occludes the respective injection portion “I”.

The sequence of successive movements of a plurality of injection valves 4 is suitable to sequentially move a plurality of injection valves 4 between the opening configuration and the closing configuration.

Therefore, the method advantageously allows the technical and/or operational characteristics of the digester 100 to be defined on the basis of the type of sludge to be processed, thus enabling optimisation in biogas production by the digester 200.

The method also allows for active control of the biogas production process by regulating it while it is running, enabling improved optimisation of biogas productivity as well as the environmental and economic sustainability of the process.

In particular, the method preferably comprises a step of configuring the control unit 200, by which at least one control parameter, relating to an optimal value of a working parameter detected by the detector device 5, can be stored in a memory unit of the control unit 200.

Preferably, this step is accomplished by entering the control parameter into a user interface device, e.g. including a keyboard and/or screen (not shown in the accompanying figures).

The step of processing the working parameter is carried out by comparing the working parameter (detected by the detector device 5) and the control parameter (stored in the control unit 200), in order to drive the compressor 2 and/or the injection portions “I”, based on the comparison.

Advantageously, this comparison makes it possible to drive (via the control unit 200) the compressor 2 and/or injection portions “I” in such a way that the detected working parameter, in case it differs from the control parameter, tends over time towards the set control parameter.

Therefore, with the control unit 200 it is possible to drive a continuous and gradual optimisation of a working parameter and thus of the operation of the digester 100.

With reference to the working parameter detection step, this is preferably carried out by detecting a working parameter near a base portion 1a of the containment body 1 and a same working parameter near a top portion 1b of the containment body 1.

In other words, the detection step is performed by detecting working parameters at opposite portions of the containment volume “V” along an extension axis “X” of the digester 100.

It is therefore advantageous to detect parameters identifying the entire volume of sludge placed in the containment volume “V” of the digester 100.

For example, by detecting a sludge temperature at the base portion 1a and the top portion 1b, the recirculation element 3 can be driven to maintain a temperature gradient within the sludge below a threshold value. As a result, biogas production by the bacteria responsible for it can be maximised.

Preferably, moreover, the biogas suction step is carried out by sucking biogas from the top portion 1b of the containment body 1 and the biogas injection step is realised by injecting biogas at the base portion 1a of the containment body 1.

An injection of biogas from the base portion 1a defines rising columns of injected biogas and sludge contained in the containment volume “V”.

The step of injecting biogas is therefore aimed at achieving a movement of sludge masses within the containment volume “V”, in detail from the base portion 1a towards the top portion 1b and from the top portion 1b towards the base portion 1a (due to the weight of the sludge itself).

In other words, the biogas injection step is aimed at moving masses of sludge contained within the containment volume “V” along a circular path, schematically illustrated in FIG. 5 by the arrows “F”, extending between the base portion 1a and the top portion 1b.

Therefore, advantageously, the method allows the entire mass of sludge contained in containment volume “V” to be mixed, and thus minimises a thermal gradient between the sludge placed at the base portion 1a and the sludge placed at the top portion 1b, maximising biogas production.

Advantageously, the method also allows sludge to be mixed within the containment volume “V” in such a way as to prevent the development of any fouling, e.g. of struvite, within the containment volume “V” of the digester 100 itself.

This results in optimised productivity and maintenance costs for the digester 100.

Preferably, the method involves a step of controlling an injected biogas flow rate based on at least one working parameter detected.

Preferably, the step of detecting at least one working parameter by means of the detector device 5 is carried out by detecting working parameters at predefined time instants.

In other words, the detector device 5 is configured to detect a time trend of a respective working parameter.

Therefore, the step of driving the compressor 2 and/or the injection portions “I” is carried out on the basis of the working parameters measured at successive instants.

Advantageously, successive commands of the control unit 200 on the basis of respective successive working parameters allow continuous optimisation of the biogas production process.

Preferably, the step of driving the injection portions “I” is carried out by driving injection valves 4 associated with them.

In detail, the step of driving the injection portions “I” preferably comprises a step of moving the at least one injection valve 4 of the recirculation element 3 between an opening configuration, in which the injection valve 4 places a respective injection portion “I” in fluid communication with the compressor 2 and the containment volume “V”, and a closing configuration, in which it occludes the respective injection portion “I”.

Preferably, the step of driving the injection portions “I” is carried out by controlling a sequence of successive movements of respective injection valves 4 between the opening configuration and the closing configuration.

In other words, the step of driving the injection portions “I” comprises a step of controlling a sequential movement of several injection valves 4 associated with them.

Advantageously, such a step of controlling a sequential movement of the injection valves 4 allows optimisation of sludge mixing by injection of biogas through the injection portions “I”, defining both a vertical movement of the sludge (i.e. from the base portion 1a to the top portion 1b along the path “F”) and a movement of the sludge along a path extending around the extension axis “X” of the containment body 1.

Thus, by controlling a sequential movement of the injection valves 4, it is possible to achieve a complete revolution of the mass of sludge within the containment volume “V” of the digester 100, thus preventing the formation of fouling and/or struvite resulting from sedimentation of sludge within the containment volume “V”.

The method thus leads to a reduction in the maintenance costs of the digester 100, an increase in its service life, as well as a maximisation of biogas production.

Preferably, moreover, the step of driving the injection portions “I” is carried out by maintaining the opening configuration and/or the closing configuration for a preset duration as a function of the working parameter and/or by defining a flow rate of biogas injected into the containment volume “V” as a function of the working parameter, or the comparison between the working parameter and the control parameter.

As mentioned above, the described method is operable in particular by a digester 100 as illustrated in the appended figures and having at least the components discussed above.

In more detail, an example structure of a possible digester 100 specifically optimised to be operated using the control logic defined by the method presented above is presented below. Structurally, the digester 100 comprises a compressor 2 arranged in fluid communication with the containment volume “V” and configured to suck the biogas contained therein.

Preferably, the compressor 2 is in fluid communication with the containment volume “V” at the top portion 1b.

In other words, a connection portion between the compressor 2 and the containment volume “V” is arranged at the top portion 1b.

The aforementioned connection portion may for example be a pipe 2a, preferably made of stainless steel and fitted with a valve 2b arranged within the channel defined by the pipe 2a itself.

With reference to FIGS. 1 to 4, the digester 100 further comprises a recirculation element 3 placed in fluid communication with the compressor 2 the containment volume “V”, configured to suck biogas from the containment volume “V” and to inject the sucked biogas into the containment volume “V” receiving at least a part of the sucked biogas.

In detail, a portion of the sucked biogas can be transferred from the compressor 2 to the recirculation element 3 and a further portion of the sucked biogas can be transferred from the compressor 2 to a collection tank commonly referred to as a gasometer.

Alternatively, the sucked biogas can be fully transferred from the compressor 2 to the recirculation element 3 for a predetermined processing period, and from the compressor 2 to a gasometer for a further predetermined discharge period.

The recirculation element 3 comprises a plurality of injection portions “1” placed in fluid communication with the containment volume “V” to inject the biogas (sucked by the compressor 2) into the containment volume “V”.

Preferably, each injection portion “I” is configured to inject a flow rate of biogas that can be defined and set using the control logic defined by the method presented above.

In other words, the recirculation element 3 is configured to recirculate biogas in the containment volume “V” in such a way that the sludge contained therein is mixed.

Preferably, the injection portions “I” are arranged at the base portion 1a.

A positioning of the injection portions “I” is in particular defined by means of the method presented above during the setting-up step of a technical characteristic of the digester 100.

In detail, the injection portions “I” can be arranged at the back wall 10 or at a predetermined distance from the back wall 10.

In use, an injection of biogas from the base portion 1a defines rising columns of the injected biogas and sludge contained in the containment volume “V”.

In other words, due to the injection of biogas from the injection portions “I”, the sludge is subject to movement from the base portion 1a to the top portion 1b and, due to its weight, is subject to reverse movement from the top portion 1b to the base portion 1a.

Therefore, thanks to the arrangement of the injection portions “I” at the base portion 1a, it is possible to define a circular motion for the sludge, developing in particular along a path, illustrated schematically in FIG. 5 by the arrows “F”, extending between the base portion 1a and the top portion 1b.

By generating circular motion of the sludge along the path “F”, the compressor 2 and the recirculation element 3 achieve uniform mixing of sludge and biogas within the digester 100.

It is therefore possible to minimise a thermal gradient between the sludge arranged at the base portion 1a and the sludge arranged at the top portion 1b, ensuring an almost constant temperature of the sludge useful to maximise biogas production by the bacteria responsible.

Preferably, the injection portions “I” are evenly distributed around the containment body 1.

Advantageously, an even distribution of the injection portions “I” ensures respective sludge mixing evenly distributed in the containment volume “V”, optimising the circular motion of sludge (schematically illustrated in FIG. 5 by the arrows “F”) generated by the injection of biogas through the injection portions “I” themselves.

Preferably, the digester 100 comprises at least 2 injection portions “I”.

In accordance with an aspect of the present invention, the recirculation element 3 comprises respective injection valves 4, associated with the plurality of injection portions “I”, schematically illustrated in FIGS. 1-4. In other words, each injection portion “I” is associated with at least one respective injection valve 4.

Preferably, the injection valves 4 are made in the form of solenoid valves, even more preferably, the injection valves 4 are made of brass.

Preferably, the injection valves 4 are arranged in series and upstream of a respective shut-off valve 4a.

Each injection valve 4 is movable between an opening configuration, in which it places the respective injection portion “I” in fluid communication with the compressor 2 and the containment volume “V”, and a closing configuration, in which it occludes the respective injection portion “I”.

In accordance with an aspect of the present invention, the recirculation element 3 comprises, as illustrated in FIGS. 1-4, a mixing manifold 3a, at least one supply duct 30 and a plurality of distribution ducts 31.

Preferably, the supply duct 30 and the distribution ducts 31 are made of stainless steel.

Operationally, the supply duct 30 is configured to place the compressor 2 in fluid communication with the mixing manifold 3a.

Preferably, a valve 3b is interposed between the supply duct 30 and the mixing manifold 3a.

The mixing manifold 3a is preferably an annular tubular element and is placed in fluid communication with the distribution ducts 31, which define the injection portions “I”.

In more detail, the supply duct 30 is configured to feed the sucked biogas into the mixing manifold 3a, while the mixing manifold 3a is adapted to distribute the biogas received between the distribution ducts 31 in fluid communication with it.

Each mixing duct 31 defines a respective injection portion “I” and preferably comprises within its respective lumen at least one injection valve 4.

Thus, an opening or closing of the injection valves 4 associated with each distribution duct 31 defines a corresponding distribution of biogas between the distribution ducts 31 themselves, and thus between the injection portions “I” (defined by the distribution ducts 31).

In accordance with a particular aspect of the present invention, the digester 100 comprises at least one detector device 5, configured to detect at least one working parameter of the digester 100 itself.

The term “working parameter” refers to a parameter identifying a sludge digestion process operated by the digester 100, such as a temperature value, pH value, sludge displacement, or a value relating to the energy consumption of the digester 100. In other words, the detector device 5 is capable of detecting a physical parameter and/or a chemical parameter relating to the sludge contained in the containment volume “V”.

Preferably, the detector device 5 is configured to detect an operating parameter of the compressor 2, such as its energy consumption. In this case, the detector device 5 is connected or connectable (wired or wireless) with the compressor 2.

In accordance with an aspect of the present invention, the digester 100 comprises at least one pair of detector devices 5 configured to detect the same working parameter.

In detail, the pair of detector devices 5 comprises a first detector device 5a, arranged near the base portion 1a, and a second detector device 5b, arranged near the top portion 1b.

Advantageously, the pair of detector devices 5a and 5b, in particular their arrangement in different portions/zones of the containment volume “V” of the digester 100, allows respective working parameters to be detected at opposite portions of the digester 100, consequently allowing a collection of working parameters indicative of the entire volume of sludge arranged within the containment volume “V”, and not only of a portion proximal to the base portion 1a, the top portion 1b, or interposed between the base portion 1a and the top portion 1b.

In accordance with a particular aspect of the present invention, the digester 100 further comprises a control unit 200, connected or connectable to the detector device 5 for receiving and processing the working parameter, as illustrated in FIGS. 1-4.

The control unit 200 is also connected or connectable with the compressor 2, to drive the compressor 2 on the basis of the processed working parameter, and/or with recirculation element 3 to drive one or more of the injection portions “I” on the basis of the processed working parameter.

In other words, the control unit 200 is configured to monitor the operation of the digester 100 (by processing the working parameter detected by each detector device 5) and to drive the compressor 2 and/or the injection portions “I” on the basis of the processed working parameter, so as to strive for an optimisation of a sludge digestion process operated by the digester 100.

In other words, the control unit 200 is adapted to minimise the energy consumption of the digester 100, and/or maximise biogas production, and/or minimise the maintenance costs of the digester 100.

The control unit 200 is connected or connectable to the detector device 5 by means of appropriate electrical cables, or by means of a wireless connection.

Similarly, the control unit 200 is connected or connectable to the compressor 2 and/or the injection portions “I” by means of appropriate electrical cables, or by means of a wireless connection.

Advantageously, in the event that the detector device 5 is configured to detect the operating parameter of the compressor 2, thanks to the control unit 200 it is possible to drive the compressor 2 and/or the injection portions “I” in such a way as to optimise the energy consumption of the compressor 2 itself, or in such a way as to set a predetermined gas flow rate generated by it.

In other words, with the detector device 5 and control unit 200 it is possible to provide a sustainable digester 100 as well as maximising biogas production and sludge mixing within the containment volume “V”.

Preferably, the control unit 200 comprises a memory unit comprising at least one control parameter.

Even more preferably, the digester 100 is connected or connectable with a user interface device (not shown in the accompanying figures), so that an operator can arbitrarily set the control parameter.

The control parameter can for example be an optimal sludge temperature, or an optimal sludge pH, for biogas production by the bacteria responsible, as well as a desired compressor 2 energy consumption value.

With reference to the control parameter, the control unit 200 is configured to process the working parameter by performing a comparison between the working parameter detected by the detector device 5 and the control parameter.

The control unit 200 is also configured to drive the compressor 2 and/or one or more of the injection portions “I”, based on the comparison between the working parameter and the control parameter.

Thus, thanks to the detector device 5 and thanks to the control unit 200, it is advantageously possible to verify correct operation of the digester 100 and, if this operation can be optimised in light of the comparison between the working parameter and the control parameter, to drive the compressor 2 and/or the injection portions “I” in such a way as to obtain a working parameter as close as possible to the respective control parameter. In other words, it is possible to strive for continuous optimisation of the sludge digestion process for producing biogas.

Preferably, the control unit 200 is also configured to detect and process parameters identifying wear of the digester 100, and to make available to an operator, e.g. by means of a user interface device (not illustrated in the accompanying figures), data useful for predictive maintenance of the digester 100, or its components.

With reference to the injection valves 4, associated with the injection portions “I”, the control unit 200 is connected or connectable to at least one injection valve 4, preferably the control unit 200 is connected or connectable to each injection valve 4.

In detail, the control unit 200 is configured to drive a respective injection valve 4 according to the working parameter of the digester 100 detected by the detection device 5.

In more detail, the control unit 200 is connected or connectable with a respective injection valve 4 to drive, based on the operating parameter, a movement of the same between the opening configuration and the closing configuration.

Preferably, the control unit 200 is connected or connectable with at least some of the injection valves 4, preferably with all of the injection valves 4, to drive a sequence of successive movements of respective injection valves 4 between the opening configuration and the closing configuration.

An opening/closing sequence of the injection valves 4 is preferably set by an operator via a user interface device.

Advantageously, such a sequence of movements of the injection valves 4 allows not only a generation of a circular motion of the sludge (schematically illustrated in FIG. 5 by the arrows “F”), but also generation of a movement of the sludge around an extension axis “X” of the digester 100, consequently obtaining a complete revolution of the mass within the containment volume “V” of the digester 100.

1. Method for the optimisation and control of biogas production processes comprising the steps of:
providing a digester (100) comprising a containment body (1) internally defining a containment volume (V) intended to contain sludge and biogas;
filling the containment volume (V) with sludge;
providing a recirculation element (3);
installing the recirculation element (3) so that said recirculation element (3) is in fluid communication with said containment volume (V) and said compressor (2);
sucking from the containment volume (V) a part of the biogas produced and/or contained in the sludge;
feeding at least part of the sucked biogas into the recirculation element (3);
injecting, in a sequential logic, said part of the sucked biogas within the containment volume (V) through respective injection portions (I) of the recirculation element (3);
detecting at least one working parameter of the digester (100) by means of at least one detector device (5);
transmitting the working parameter to a control unit (200);
processing the working parameter by means of the control unit (200);
driving by means of the control unit (200) the activation of one or more injection portions (I) based on said processed working parameter.

2. The method according to claim 1, further comprising the steps of:
acquiring information on a type of sludge intended to be contained within the containment volume (V) and/or on a volume of sludge intended to be contained within the containment volume (V);
setting a technical characteristic of said digester (100) based on said acquired information.

3. The method according to claim 2, wherein said technical characteristic relates to one or more technical characteristics selected from:
a number of injection portions (I);
a mutual positioning between the injection portions (I);
a positioning of the injection portions (I) within the containment volume (V).

4. The method according to claim 2, wherein said step of setting a technical characteristic of the digester (100) based on the acquired information comprises storing in a memory unit of the control unit (200);
a sequence of commands, to move at least one injection valve (4) of the recirculation element (3) according to a predefined sequence of movements between an opening configuration, wherein said injection valve (4) allows injecting biogas through the respective injection portion (I), and a closing configuration, wherein said injection valve (4) occludes the respective injection portion (I), and/or
a sequence of successive movements of a plurality of injection valves (4) between said opening configuration and said closing configuration.

5. The method according to claim 1, further comprising a step of storing in a memory unit of the control unit (200) at least one control parameter and wherein said step of processing the working parameter is carried out by comparing said working parameter and said control parameter, so as to drive the injection portions (I) based on said comparison.

6. The method according to claim 1, wherein the step of detecting at least one working parameter is carried out by detecting working parameters at predetermined time instants, and wherein the step of driving the injection portions (I) is carried out based on said working parameters.

7. The method according to claim 1, wherein the step of detecting at least one working parameter is carried out by detecting a working parameter near a base portion (1a) of the containment body (1) and a same working parameter near a top portion (1b) of the containment body (1).

8. The method according to claim 1, wherein the step of driving one or more of the injection portions (I) based on said processed working parameter comprises:
moving according to said processed working parameter at least one injection valve (4) of the recirculation element (3) between an opening configuration, wherein it allows injecting biogas through the respective injection portion (I), and a closing configuration, wherein it occludes said injection portion (I), and/or
commanding according to said processed working parameter a sequence of successive movements of respective injection valves (4) between said opening configuration and said closing configuration.

9. The method according to claim 8, wherein said step of driving the injection portions (I) is performed by maintaining the opening configuration and/or the closing configuration for a preset duration according to the working parameter and/or by defining a flow rate of injected biogas within the containment volume (V) according to the processed working parameter.

10. The method according to claim 1, wherein the step of sucking biogas is performed by sucking biogas from a top portion (1b) of the containment body (1) of the digester (100) and wherein the step of injecting biogas is performed by injecting biogas at a base portion (1a) of the containment body (1) of the digester (100).

11. The method according to claim 1 wherein the step of detecting at least one working parameter is performed by detecting one or more of:
at least one chemical parameter of sludge and/or biogas contained within said containment volume (V);
at least one physical parameter of sludge and/or biogas contained within said containment volume (V).

12. Digester (100) for the purification and production of biogas from sludge, configured to operate a method according to claim 1, comprising:
a containment body (1) extending between a base portion (1a) and a top portion (1b) and internally defining a containment volume (V) intended to contain sludge and biogas;
at least one compressor (2) in fluid communication with said containment volume (V) and configured to suck a biogas contained in said containment volume (V);
a recirculation element (3) placed in fluid communication with said compressor (2) to receive at least a portion of the sucked biogas and comprising a plurality of injection portions (I) placed in fluid communication with said containment volume (V) to inject said sucked biogas within said containment volume (V);
at least one detector device (5), configured to detect at least one working parameter of said digester (100), and
a control unit (200) connected or connectable to said detector device (5) to receive and process said working parameter;
said control unit (200) also being connected or connectable with said compressor (2), to drive said compressor (2) according to said processed working parameter, and/or with said recirculation element (3) to drive one or more of said injection portions (I) according to said processed working parameter.

13. The digester according to claim 12, wherein said recirculation element (3) comprises respective injection valves (4), associated with the plurality of injection portions (I), and wherein said control unit (200) is configured to drive one or more injection valves (4) according to said processed working parameter.

14. The digester (100) according to claim 12 wherein each injection valve (4) is movable between an opening configuration, wherein it places the injection portion (I) in fluid communication with said compressor (2) and with said containment volume (V), and a closing configuration, wherein it occludes said injection portion (I) of said recirculation element (3),
said control unit (200) being operatively connected to at least one injection valve (4), to drive a movement of said injection valve (4) between said opening configuration and said closing configuration based on the processed working parameter, and/or said control unit

(200) is operatively connected to injection valves (4) to drive a sequence of successive movements of respective injection valves (4) between the opening configuration and the closing configuration.

15. The digester (100) according to claim 12, comprising at least one pair of detector devices (5) configured to detect the same working parameter as the digester (100), said pair of detector devices (5) comprising a first detector device (5a) arranged near the base portion (1a) and a second detector device (5b) arranged near the top portion (1b).
